(19) United States
(12) Patent Application Publication
(10) Pub. No.: US 2015/0141651 A1
(43) Pub. Date: May 21, 2015

(54) HEXAHYDROINDENOPYRIDINE DERIVATIVES

(71) Applicant: Boehringer Ingelheim International GmbH, Ingelheim am Rhein (DE)

(72) Inventors: Jean-Nicolas DESROSIERS, Southbury, CT (US); Zhibin LI, Berlin, CT (US); Bo QU, Brookfield, CT (US); Jolaine SAVOIE, Danbury, CT (US); Chris Hugh SENANAYAKE, Brookfield, CT (US); Xudong WEI, Ridgefield, CT (US); Xingzhong ZENG, New Milford, CT (US)

(21) Appl. No.: 14/547,463

(22) Filed: Nov. 19, 2014

Related U.S. Application Data
(60) Provisional application No. 61/906,653, filed on Nov. 20, 2013.

(51) Int. Cl. C07D 401/06; C07D 221/06

(52) U.S. Cl. CPC C07D 401/06; C07D 221/06
USPC 546/111

(57) ABSTRACT

This application relates methods of making compounds A and B:

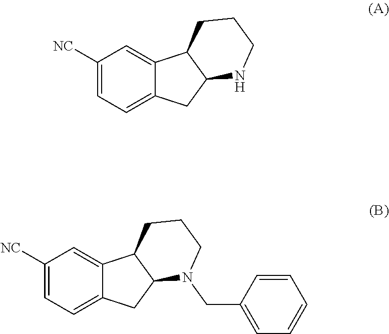 [SMILES: N#Cc1ccc2c(c1)[C@H]1CCCN[C@H]1C2 ; N#Cc1ccc2c(c1)[C@H]1CCCN(Cc3ccccc3)[C@H]1C2]

Compounds A and B are useful as intermediates for the preparation of pharmaceutically active compounds such as 11-β-hydroxysteroid hydrogenase type 1 (11-β-HSD1) inhibitors.

### TECHNICAL FIELD

This application relates to hexahydroindenopyridine derivatives. The hexahydroindenopyridine derivatives of the invention are useful as intermediates for the preparation of pharmaceutically active compounds such as 11-β-hydroxysteroid hydrogenase type 1 (11-β-HSD1) inhibitors.

### BACKGROUND OF THE INVENTION

Aryl- and heteroarylcarbonyl derivatives of hexahydroindenopyridines are useful as inhibitors of 11-β-hydroxysteroid hydrogenase type 1 (“11-β-HSD1”) and for treatment of disorders associated with 11-β-HSD1 activity including, for example, diabetes mellitus (e.g., type II diabetes), obesity, symptoms of metabolic syndrome, glucose intolerance, hyperglycemica (see, e.g., WO 2011/057054).

The aryl- and heteroarylcarbonyl derivatives of hexahydroindenopyridines can be prepared, for example, from nitrile-substituted hexahydroindenopyridines as described in WO 2011/057054. In one method described in WO 2011/057054, the intermediate (4aR,9aS)-2,3,4,4a,9,9a-hexahydro-1H-indeno[2,1-b]pyridine-6-carbonitrile (A) is allowed to react with 1H-benzoimidazole-5-carboxylic acid followed by reaction with hydrogen chloride to provide the 11-β-HSD1 inhibitor (4a-R,9a-S)-1-(1H-benzoimidazole-5-carbonyl)-2,3,4,4a,9,9a-hexahydro-1H-indeno[2,1-b]pyridine-6-carbonitrile (F) as depicted below:

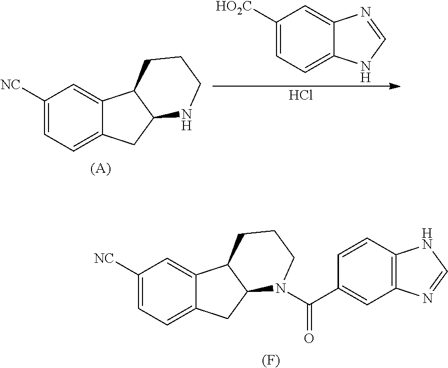

Some methods of preparing compound A are described in WO 2011/057054 and WO/2013/025664. The described methods include preparing the racemic form of compound A followed by a resolution step. Such methods necessarily require discarding the undesired enantiomer which adds to the cost of the final product.

Thus, there is a need, for improved processes for making compound A, and derivatives thereof. Such improvements in making compound A, and derivative thereof, will allow for more efficient preparation of aryl- and heteroarylcarbonyl derivatives of hexahydroindenopyridines inhibitors, particularly for large-scale production.

### BRIEF SUMMARY OF THE INVENTION

The invention relates, in one embodiment, to methods of making compounds A and B:

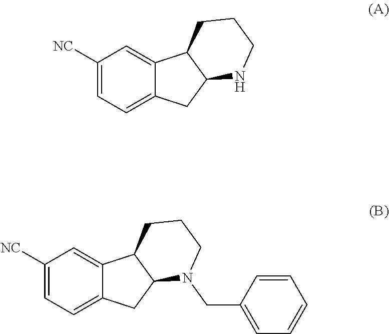 [SMILES: N#Cc1ccc2c(c1)[C@H]1CCCN[C@H]1C2 ; N#Cc1ccc2c(c1)[C@H]1CCCN(Cc3ccccc3)[C@H]1C2]

In a first embodiment, the invention relates to a method of making a compound B, the process comprising:

reacting compound C or a salt of compound C (“C⁺X⁻”):

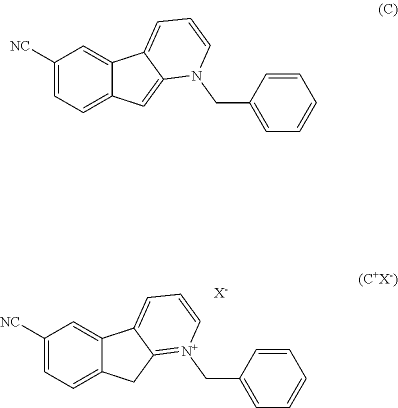

with a transition metal complex in the presence of hydrogen to provide compound B (“the asymmetric hydrogenation step”); wherein
X⁻ is an anion selection from the group consisting of Cl⁻, Br⁻, I⁻, BF₄⁻, PF₆⁻, and MeSO₃⁻.

In a second embodiment, the invention relates to a method of making a diastereomeric salt of compound B (“B-EA”), the process comprising:

reacting compound B with an enantiomeric organic acid (“EA”) to provide compound B-EA,
wherein the enantiomeric organic acid is selected from the group consisting of D-DTTA, D-DBTA, and D-tartaric acid.

In a third embodiment, the invention relates to a method of making compound A, the process comprising reacting compound B-EA with base followed by reaction with a debenzylating reagent to provide compound A.

In a fourth embodiment, the invention relates to a method of making compound A, the process comprising:

preparing compound B by the asymmetric hydrogenation of C or C⁺X⁻ as described in the first embodiment above,
reacting compound B with an enantiomeric organic acid to provide compound B-EA as described in the second embodiment above, and
reacting compound B-EA with base followed by reaction with a debenzylating reagent to provide compound A as described in the third embodiment above,
wherein the enantiomeric organic acid is selected from the group consisting of D-DTTA, D-DBTA, and D-tartaric acid.

In a fifth embodiment, the invention relates to a method of making compound A (crude), the process comprising:

preparing compound B by the asymmetric hydrogenation of C or C⁺X⁻ as described in the first embodiment above, and
reacting compound B with a debenzylating reagent to provide compound A (crude).

In a sixth embodiment, the invention relates to a method of making an enantiomeric salt of compound A (A-EA), comprising:

preparing compound A as described in the fifth embodiment above, and
reacting compound A with an enantiomeric organic acid to provide compound A-EA, wherein the enantiomeric organic acid is selected from the group consisting of D-DTTA, D-DBTA, and D-tartaric acid.

In another embodiment, the invention relates to a method of making compound A, the process comprising reacting compound B-EA with base followed by reaction with a debenzylating reagent to provide compound A.

The invention still further relates to the D-DTTA salt of compound B.

### DETAILED DESCRIPTION OF THE INVENTION

### Abbreviations

ACE-Cl=1-chloroethyl chloroformate
B(C₆H₅)₄⁻=tetraphenylborate
B[3,5-(CF₃)₂C₆H₃]₄⁻ (BArF⁻)=tetrakis[(3,5-trifluoromethyl)phenyl]borate
BF₄⁻=tetrafluoroborate
DCM=dichloromethane
D-DBTA=di-benzoyl-D-tartaric acid
DMSO=dimethyl sulfoxide
D-DTTA=di-toluoyl-D-tartaric acid
ee=enantiomeric excess
er=enantiomeric ratio
EtOAc=ethyl acetate
[IrCl(COD)]₂=chloro(1,5-cyclooctadiene)iridium (I) dimer
LDA=lithium diisopropylamide
MeCN=acetonitrile
NaBArF=sodium tetrakis[(3,5-trifluoromethyl)phenyl]borate
PF₆⁻=hexafluorophosphate
PMHS=polymethylhydrosilane
SbF₆⁻=hexafluoroantimonate
t-Bu=tertiary butyl group
TfO⁻=trifluoromethanesulfonate
THF=tetrahydrofuran

All terms used herein in this specification, unless otherwise stated, shall be understood in their ordinary meaning as known in the art. For example, “(C₁-C₆)alkoxy” or “O(C₁-C₆)alkyl” is a (C₁-C₆)alkyl with a terminal oxygen, such as methoxy, ethoxy, propoxy, and butoxy. All alkyl, alkenyl, and alkynyl groups shall be understood as being branched or unbranched where structurally possible and unless otherwise specified. Other more specific definitions are as follows:

The term “alkyl” refers to both branched and unbranched alkyl groups. It should be understood that any combination term using an “alk” or “alkyl” prefix refers to analogs according to the above definition of “alkyl”. For example, terms such as “alkoxy”, “alkylhio” refer to alkyl groups linked to a second group via an oxygen or sulfur atom. “Alkanoyl” refers to an alkyl group linked to a carbonyl group (C═O).

In all alkyl groups or carbon chains, one or more carbon atoms can be optionally replaced by heteroatoms such as O, S or N. It shall be understood that if N is not substituted then it is NH. It shall also be understood that the heteroatoms may replace either terminal carbon atoms or internal carbon atoms within a branched or unbranched carbon chain. Such groups can be substituted as herein above described by groups such as oxo to result in definitions such as but not limited to: alkoxycarbonyl, acyl, amido and thioxo. As used herein, “nitrogen” and “sulfur” include any oxidized form of nitrogen and sulfur and the quaternized form of any basic nitrogen. For example, for a —S—(C₁-C₆)alkyl radical, unless otherwise specified, shall be understood to include —S(O)— (C₁-C₆)alkyl and —S(O)₂—(C₁-C₆)alkyl.

The term “olefin” as used herein refers to an unsaturated hydrocarbon containing carbon atoms linked by a double bond (i.e., an alkene) such as, for example, ethylene, propene, 1-butene, 2-butene, styrene, norbornadiene, or cyclooctadiene. The term “diolefin” refers an unsaturated hydrocarbon containing two pairs of carbon atoms linked by double bonds, e.g., norbornadiene, or cyclooctadiene.

The compounds of the invention may be made using the general synthetic methods described below or in the Examples, which also constitute parts of the invention.

As noted above, one embodiment of the invention relates to a method for making compound B by the asymmetric hydrogenation of compound C or C⁺X⁻ in the presence of a transition metal catalyst.

Compound C can be prepared by the method described in WO/2013/025664, and compound C⁺X⁻ can be prepared treating the compound C with an appropriate acid, e.g., hydrochloric acid or hydrobromic acid.

In one embodiment, the transition metal complex used to carry out the asymmetric hydrogenation is of the type described in WO/2011/056737 and depicted below as formula (IIa):

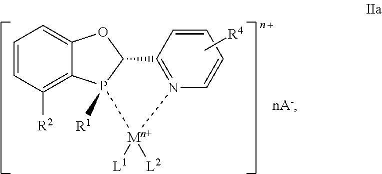

wherein
M is a transition metal selected from Rh and Ir;
A⁻ is a counter anion selected from the group consisting of chloro, bromo, halo, BF₄⁻, SbF₆⁻, TfO⁻, B(C₆H₅)₄⁻, B[3,5-(CF₃)₂C₆H₃]₄⁻, (BArF)⁻, or PF₆⁻;
n is the oxidation state of the transition metal M;
L¹ and L² are each olefins, or L¹ and L² together represent a diolefin;
R¹ is —(C₁-C₆)alkyl;
R² is H, —O(C₁-C₆)alkyl or —(C₁-C₆)alkyl; and
R⁴ is selected from —O(C₁-C₆)alkyl and —(C₁-C₆)alkyl.

In one embodiment, the hydrogenation step comprises a metal complex of formula (IIa) wherein:

n is 1;
R¹ is t-Bu,

### R² is —OCH₃;

R⁴ is —OCH₃ attached to the ortho-position of the pyridyl ring; and
L¹ and L² together represent a diolefin selected from norbornadiene and cyclooctadiene.

In another embodiment, the hydrogenation step comprises a metal complex of formula (IIa) as described in the embodiment immediately above, wherein M is Ir, and

L¹ and L² together represent cyclooctadiene.

In another embodiment, the hydrogenation step comprises a metal complex of formula (IIa) as described in the embodiment immediately above, wherein A⁻ is Br⁻.

The transition metal catalyst may be prepared as shown in Scheme 1.

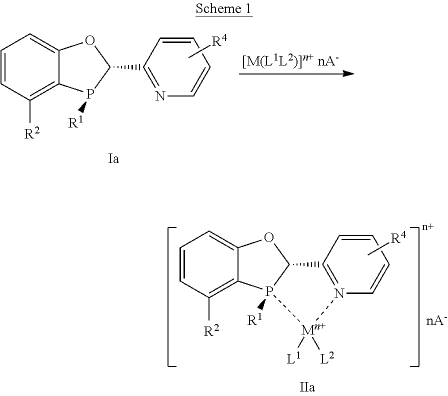

As illustrated in Scheme 1, reaction of a chiral ligand of formula ha with a transition metal salt [M(L¹L²)]ⁿ⁺nA⁻, in a suitable solvent, provides the compound of formula IIa.

The transition metal catalyst is typically prepared in situ and used without isolation. In one embodiment, the transition metal complex is prepared by:

reacting [IrCl(COD)]₂ with chiral ligand Ia in organic solvent to form a first transition metal complex solution; and
reacting the first transition metal complex solution with I₂ to provide the transition metal complex.

Without being limited by theory, the inventors of the subject application believe that the transition metal complex first reacts with H₂ to form an activated catalyst system before the asymmetric hydrogenation of C or C⁺X⁻ occurs.

In one embodiment, the transition metal catalyst used in the asymmetric hydrogenation is reacted with H₂ in the absence of compound C or C⁺X⁻ to form an activated catalyst, and the activated catalyst is used to carry out the asymmetric hydrogenation of compound C or C⁺X⁻ with H₂ to provide compound B.

In another embodiment, the transition metal catalyst used in the asymmetric hydrogenation is reacted with H₂ in the presence of compound C or C⁺X⁻ to form an activated catalyst, which activated catalyst is used to carry out the asymmetric hydrogenation of compound C or C⁺X⁻ with H₂ to provide compound B.

In some embodiments, the asymmetric hydrogenation of converts at least about 95% of C or C⁺X⁻ into product, and, in some embodiments, at least about 99% of C or C⁺X⁻ into product. The products formed from the asymmetric hydrogenation step include the desired product (compound B) and also minor amounts of other isomers and impurities. In one embodiment, the asymmetric hydrogenation step provides compound B in enantiomeric excess of at least about 65%.

Because the product formed from the asymmetric hydrogenation step may contain minor amounts of other isomers and by-products, the product is sometimes referred to herein as compound B (crude). Methods of removing the minor isomers and other by-products are described below.

Scheme 2 depicts one method of making compound Ia.

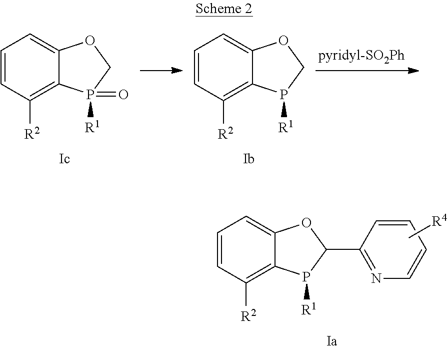

As illustrated in Scheme 1, reduction of the phosphine oxide Ic under suitable conditions provides Ib. Compound Ib may be further converted to a compound of formula Ia by the reaction with desired pyridyl sulfone of formula:

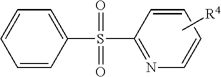

in a suitable solvent in the presence of a suitable base to provide a compound of formula Ia. The synthesis of compound Ic is described in WO 2011/056737 and the Examples.

Alternatively, the compounds of formula Ia may be prepared by reacting a compound of formula Ic with a suitable deprotonating agent (e.g., LDA) following by reaction with the desired pyridyl sulfone to provide a pyridyl phosphine oxide, and reacting the pyridyl phosphine oxide with a suitable reducing agent (e.g., PMHS) to provide a compound of formula Ia.

Preferably, the chiral ligand Ia used in the asymmetric hydrogenation is of formula Ma:

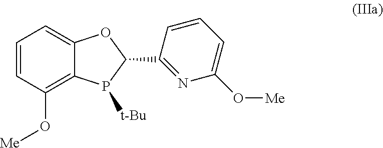 [SMILES: COc1cccc([C@H]2Oc3cccc(OC)c3[P@]2C(C)(C)C)n1]

The asymmetric hydrogenation step is carried out with an over pressure of hydrogen sufficient to carry out the hydrogenation. Typically, the hydrogen overpressure is from about 25 PSI to about 1000 PSI; or from about 25 PSI to about 500 PSI; or from about 300 PSI to about 500 PSI. It will be understood that the hydrogen overpressure can vary during the course of the hydrogenation step.

The asymmetric hydrogenation step is carried out at a time and temperature sufficient to carry out the hydrogenation. Typically, time for carrying out the asymmetric hydrogenation is from about 0.5 hr to about 48 hr; or about 24 hr. Typically, the temperature for carrying out the hydrogenation step is from about 0° C. to about 200° C.; or from about 25° C. to about 150° C.; or from about 25° C. to about 75° C.

The asymmetric hydrogenation step may be carried out in any media which allows for the hydrogenation to proceed. Typically, the asymmetric hydrogenation is carried out in a liquid diluent under heterogeneous or homogenous conditions. Preferably, the asymmetric hydrogenation is carried out under conditions whereby at least one of the transition metal complex and compound C is dissolved in a liquid diluent (e.g., THF or other suitable solvent).

In one embodiment, the invention relates to a method of carrying out the asymmetric hydrogenation step, the method comprising:

reacting compound C⁺X⁻ with H₂ in the presence of a transition metal complex to provide compound B (crude); wherein
the transition metal complex is prepared by reacting a solution of [IrCl(COD)]₂ in an organic solvent with compound IIIa to form a first mixture;
reacting the first mixture with I₂ to provide the transition metal complex.

In another embodiment, the invention relates to a method of carrying out the asymmetric hydrogenation step as described in the embodiment immediately above, wherein X⁻ in the compound C⁺X⁻ is Br⁻.

In one embodiment, the invention relates to a method of carrying out the asymmetric hydrogenation step, the method comprising:

reacting compound C with H₂ in the presence of a transition metal complex to provide compound B (crude); wherein
the transition metal complex is prepared by reacting a solution of [IrCl(COD)]₂ in an organic solvent with compound IIIa to form a first mixture; and
reacting the first mixture with I₂ to provide the transition metal complex.

As noted above, the asymmetric hydrogenation step provides compound B in enantiomeric excess of at least about 65%. The increased yield of B relative to other enantiomers in the asymmetric hydrogenation step improves the efficiency of the processes of the invention and further reduces undesired waste streams.

In one embodiment, compound B can be separated (resolved) from the other isomers, by-products and/or impurities by contacting B (crude) formed in the asymmetric hydrogenation step with an appropriate enantiomeric organic acid (EA) to provide a diastereomeric salt of B (B-EA). Compound B-EA formed by the process of the invention has, in one embodiment, an ee of at least about 95%, and in another embodiment, an ee of at least about 99%. Compound B-EA can then be reacted with base and a suitably debenzylating agent to provide compound A.

In another embodiment, compound B (crude) formed in the asymmetric hydrogenation step is reacted with a debenzylating agent to provide compound A. Compound A formed by debenzylating compound B (crude) is sometimes referred to as compound A (crude), because the product (comprising compound A) may contain minor amounts of impurities derived from the impurities present in compound B (crude). Compound A (crude) can be separated (resolved) from the other isomer and/or other by-products or impurities by contacting compound A (crude) with an appropriate enantiomeric organic acid (EA) to provide compound A-EA. Compound A-EA can then be reacted with base to provide compound A in free-base form.

Nonlimiting examples of enantiomeric acids (EAs) useful for resolving compound B and compound A include D-DTTA, D-DBTA and D-tartaric acid.

In one embodiment, the invention relates to a method of making the D-DTTA salt of compound B comprising reacting compound B (or B (crude)) formed in the asymmetric hydrogenation step with D-DTTA to provide the D-DTTA salt of compound B.

If desired, compound B-EA may be reacted with suitable base to provide the compound B in free-base form. Nonlimiting examples of suitable bases useful for preparing the free-base form of compound B from compound B-EA, or for preparing the free base form of compound A from compound A-EA, include NaOH, Na₂CO₃ and triethylamine.

A non-limiting example of a useful debenzylating agent is 1-chloroethyl chloroformate.

As discussed above, compound A is useful for making compound F (see, e.g., WO 2011/057054 and WO/2013/025664):

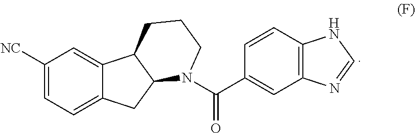 [SMILES: N#Cc1ccc2c(c1)[C@H]1CCCN(C(=O)c3ccc4[nH]cnc4c3)[C@H]1C2]

For example, 1H-benzoimidazole-5-carboxylic acid can be reacted with the free base form of compound A to provide compound F; or compound A-EA can be treated with base followed by reaction with 1H-benzoimidazole-5-carboxylic acid to provide compound F. The reaction can be carried out under conditions described in WO/2013/025664. For example, the reaction mixture is carried out using triethylamine with a slight molar excess of T3P (propane phosphoric acid anhydride) or 1-ethyl-3-(3-dimethyllaminopropyl)carbodiimide hydrochloride (EDC.HCl) and 1 molar equivalent of hydroxybenzotriazole hydrate (HOBt.H₂O). Following workup the product can be treated with EtOAc to provide an EtOAc solvate of F. If desired, the EtOAc solvate of F can be treated with hydrochloric acid in ethanol to provide an HCl salt of compound F (see WO/2013/025664).

In another embodiment, the invention relates to a method of making compound F, the method comprising: reacting the free-base form of compound A prepared by any of the embodiments above with 1H-benzoimidazole-5-carboxylic acid followed to provide compound F.

In another embodiment, the invention relates to a method of making compound F, the method comprising:

reacting the compound A-EA prepared by any of the embodiments with base to provide compound A,
and reacting compound A obtained in the step above with 1H-benzoimidazole-5-carboxylic acid to provide compound F.

In another embodiment, the invention relates to a method of making an EtOAc solvate of compound F, the method comprising: contacting compound F prepared according to any of the 2 embodiments immediately above with EtOAc to provide the EtOAc solvate of F.

If another embodiment, the invention relates to a method of making a hydrogen chloride (HCl) salt of compound F, the method comprising: contacting the EtOAc solvate of F prepared according to the embodiment immediately above with hydrochloric acid in ethanol to provide the HCl salt of compound F.

### General Synthetic Methods

Optimum reaction conditions and reaction times may vary depending on the particular reactants used. Unless otherwise specified, solvents, temperatures, pressures, and other reaction conditions may be readily selected by one of ordinary skill in the art. Specific procedures are provided in the Examples section. Typically, reaction progress may be monitored by thin layer chromatography (TLC), if desired, and intermediates and products may be purified by chromatography on silica gel and/or by recrystallization. The examples which follow are illustrative and, as recognized by one skilled in the art, particular reagents or conditions could be modified as needed for individual compounds without undue experimentation.

All of the compounds of the invention may prepared by the methods described above and in the Examples section below.

### EXAMPLES

### Example 1

### Synthesis of Compound IIa

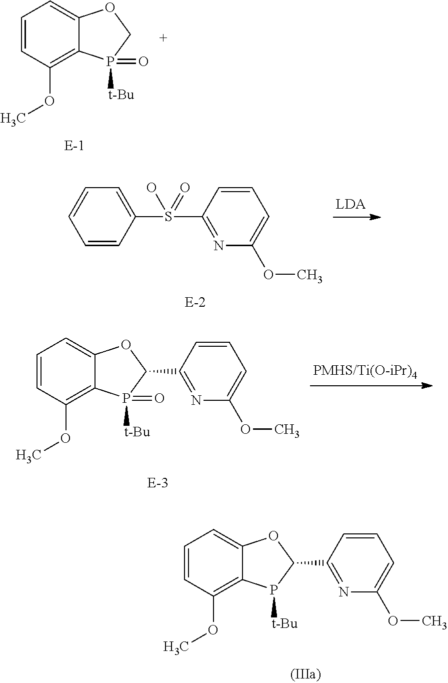

Step 1. Compound E-1 (10.0 g, 41.63 mmol), 2-methoxy-6-pyridinyl-sulphone (E-2) (10.48 g, 42.04 mmol) and anhydrous THF (300 mL) are charged to a reaction vessel under N₂. The reactor is placed in a dry ice acetone bath, and the contents of the reactor are cooled to below −70° C. LDA (2 M in THF/ethylbenzene) (62.5 ml, 124.88 mmol) is slowly added to the reaction vessel while maintaining an internal temperature below −65° C. The resulting mixture is stirred for an additional 1 hr. at −70° C. and slowly treated with MeOH (50 mL) while maintaining an internal temperature below −65° C. The contents of the reactor are warmed to room temperature, stirred for 2 hrs., and concentrated under reduced pressure. The resulting residue is treated with 50 ml water and 100 mL CH₂Cl₂ to provide a biphasic mixture. The organic layer is collected, and the aqueous layer is further extracted with 2×100 mL of CH₂Cl₂. The combined organic layer is washed with 50 mL water, dried over Na₂SO₄, and concentrated. The resulting residue is then purified by silica gel chromatography using 100% EtOAc as the eluent. The eluted fraction containing product was concentrated and further purified by recrystallization from CH₂Cl₂/hexanes at 0° C. to provide the pyridyl phosphine oxide (E-3) as a white solid. Yield: 75% yield of the phosphine oxide.

Step 2. Compound E-3 from Step 1 (6.0 g, 17.27 mmol) and degassed THF (100 mL) are charged to a reaction vessel. The mixture is then treated with polymethylhydrosiloxane (PMHS) (17.5 ml) and Ti(OiPr)₄ (15.19 ml, 51.82 mmol), and the contents of the reaction vessel are heated to 65° C. under a low flow of N₂. The reaction mixture is stirred at 65° C. for 14 h under N₂ atmosphere, slightly cooled, and concentrated in the reaction vessel under reduced pressure. The reaction vessel is placed in an ice bath (0° C.) and slowly treated with degassed aqueous NaOH (30 wt %). The first 0.5-1 mL of NaOH aqueous solution is added very slowly due to rapid evolution of H₂. The mixture is heated to 60° C. and held at 60° C. for about 1 hr. The mixture is then extracted with 5×100 mL of degassed anhydrous MTBE under argon protection. The combined organic layer is filtered through a neutral alumina pad with anhydrous MgSO₄ on the top and concentrated under reduced pressure to provide Ma as a white solid. Yield: 85% yield. ¹H NMR (500 MHz, CDCl₃) δ1.12 (d, J=12, 1H); 3.80 (s, 3H), 3.89 (s, 3H); 5.92 (s, 1H); 6.48 (dd, J=8.0, 3.5 Hz, 1H); 6.51 (d, J=8.0 Hz, 1H); 6.72 (d, J=8.0 Hz, 1H); 6.74 (t, J=1.0 Hz, 1H); 7.28 (m, 1H); 7.42 (t, J=8.0 Hz, 1H).

### Example 2

### Preparation of D-DTTA Salt of Compound B

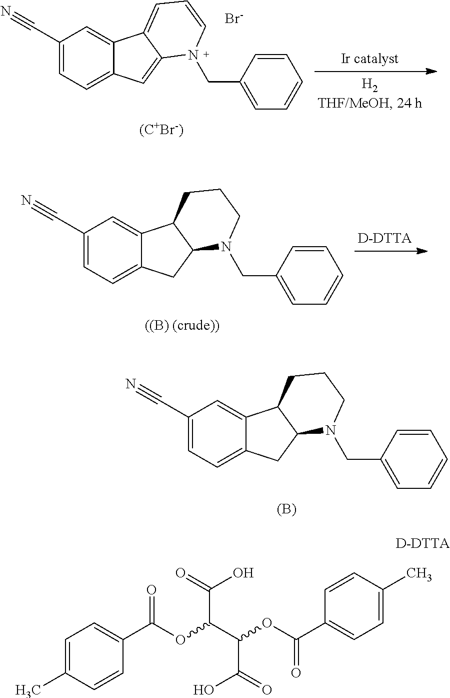

### Step 1: Asymmetric Hydrogenation

In a glove box, [IrCl(COD)]₂ (1.294 g. 1.927 mmol) and 1.92 g (5.78 mmol) of chiral ligand IIIa are charged into a 1 L Schlenk flask equipped with a stir bar and treated with anhydrous THF (200 mL). The resulting clear yellow solution is stirred at 25° C. for 20 to 30 min, treated with a solution of a solution of I₂ (2.45 g, 9.635 mmol) in anhydrous THF (100 mL), and stirred at about 25° C. for 5 min to provide a red-brown catalyst solution.

A solution of compound C⁺Br⁻ (700 g; 1.927 mol) in a mixture of anhydrous THF (7336 mL) and anhydrous MeOH (2545 mL) is charged to a 2 L hydrogenation reactor. The contents of the reactor are stirred at 100 RPM, and the reactor is purged with N₂ (3×100 psi) and H₂ (3×50 psi). The catalyst solution is charged to the reactor via Teflon tubing under N₂ pressure. The reaction mixture is purged 3 times with 50 psi H₂, pressurized to 350 psi H₂, heated to 50° C., and hydrogenated under 450 psi H₂ for 24 h. The reaction mixture is then cooled to 23° C. with stirring (300 rpm), vented, purged with N₂, and vented again (100 rpm). The resulting slurry is transferred to a 20 L reactor, concentrated under vacuum to minimum stirrable volume, treated with toluene (6.3 L), and cooled to 25° C. The mixture is then treated with 10% NaOH (2.8 L) and stirred for 10 min. The organic layer is collected, washed with water (2.8 L), and filtered through a Charcoal Cartridge to provide B (crude) as dry solid (94% yield; er 85/15). ¹H NMR (500 MHz, CDCl₃) δ 1.45-1.65 (m, 3H), 1.8-1.85 (m, 1H), 2.35-2.45 (m, 1H), 2.5-2.55 (m, 1H), 2.79 (dd, J=16.0 and 6.0 Hz, 1H), 3.1-3.15 (m, 2H), 3.42 (q, J=6.0 Hz, 1H), 3.52 (d, J=14.0 Hz, 1H), 3.77 (d, J=14.0 Hz, 1H), 7.2-7.3 (m, 6H), 7.4-7.45 (m, 2H).

### Step 2: Preparation of D-DTTA Salt of Compound B

In a 20 L reactor, a solution of B (crude) (457 g; 1.585 mol) in toluene (1 L) is concentrated to a minimum volume. Acetonitrile (2.3 L) is added and the mixture is concentrated to minimum stirrable volume. Acetonitrile (2.3 L) and THF (4.1 L) are added and the mixture is heated to 60° C. The mixture is then treated with acetonitrile (3.7 L) and a solution of di-toluoyl-D-tartaric acid (520 g, 0.85 eq, 1.32 mol) in acetonitrile (1.8 L). The mixture is stirred at 60° C. for 1 h, cooled to 25° C. over 2 h, and held at 25° C. for 2 h. The solids are collected by filtration, washed with MeCN/THF (1 L, 2/1 vol/vol), and dried under vacuum to provide the D-DTTA salt of B as a solid: Yield: 855 g, 80% yield. The structure was confirmed by HPLC, LC-MS, and NMR in DMSO-d₆. Chiral HPLC showed >99% ee. The structure was confirmed by HPLC, LC-MS, and NMR. Chiral HPLC showed >99% ee. ¹H NMR (500 MHz, DMSO-d₆) δ 1.35-1.45 (m, 1H), 1.45-1.55 (m, 1H), 1.6-1.7 (m, 1H), 1.7-1.8 (m, 1H), 2.35-2.45 (m, 1H), 2.39 (s, 6H), 2.55-2.65 (m, 1H), 2.92 (dd, J=16.5 and 6.0 Hz, 1H), 3.18 (q, J=6.0 Hz, 1H), 3.24 (dd, J=16.5 and 6.0 Hz, 1H), 3.48 (q, J=6.0 Hz, 1H), 3.66 (d, J=13.5 Hz, 1H), 3.90 (d, J=13.5 Hz, 1H), 5.78 (s, 2H), 7.25-7.3 (m, 1H), 7.3-7.35 (m, 4H), 7.37 (d, J=8.5 Hz, 4H), 7.46 (d, J=8.0 Hz, 1H), 7.60 (s, 1H), 7.62 (d, J=8.0 Hz, 1H), 7.89 (d, J=8.5 Hz, 4H).

### Example 3

### Preparation of Compound A

The D-DTTA salt of compound B (675 g, 1 mol) is charged to a 10 L reactor and treated with toluene (4.9 L). The mixture is slowly treated with 10% NaOH (800 g) at 25° C. and stirred 25° C. for 30 min. The aqueous layer is removed, and the organic layer is collected and slowly treated with 10% NaOH (800 g) at 25° C. The mixture is then stirred at 25° C. for 30 min. The aqueous layer is removed and the organic layer is concentrated to minimum volume under vacuum. The resulting organic residue is then treated with dry toluene (2.9 L), N,N-diisopropylethylamine (34.8 mL, 200 mmol) and 1-chloroethyl chloroformate (140 mL, 1250 mmol). The resulting solution is stirred at 60° C. for 1 h, treated with additional N,N-diisopropylethylamine (34.8 mL, 200 mmol) and 1-chloroethyl chloroformate (32.3 mL, 293 mmol), and stirred at 60° C. for 1 h. The mixture is then concentrated to minimum volume, treated with MeOH (2.9 L), stirred at 50° C. for 1 h, and concentrated to minimum volume. The resulting slurry is treated with heptane (2.9 L) and cooled to 25° C. The solids are collected by filtration and rinsed with heptane (290 mL). The solids are transferred to a reactor and treated with toluene (2.9 L, 10 V) and 10% NaOH (800 g). The resulting biphasic mixture is stirred at 25° C. for 30 min. The organic layer is collected and concentrated to minimum volume under vacuum to provide compound A. A sample was taken and analyzed by HPLC, LC-MS, chiral HPLC & ¹H NMR assay with internal standard in CDCl₃ confirming the desired product A. Yield: 90% yield, >99% ee.

What is claimed is:

1. A method of making compound B: 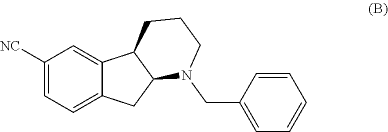 [SMILES: N#Cc1ccc2c(c1)[C@H]1CCCN(Cc3ccccc3)[C@H]1C2]
the process comprising:
reacting compound C or a salt of compound C (“C⁺X⁻”): 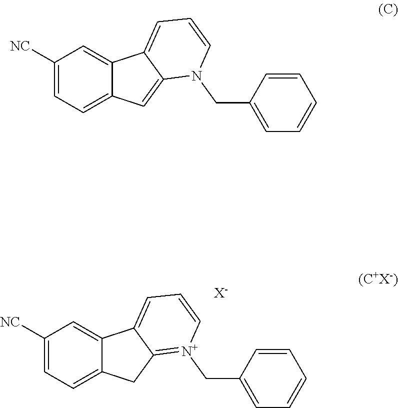
with a transition metal complex in the presence of hydrogen to provide compound B; wherein
X⁻ is an anion selection from the group consisting of Cl⁻, Br⁻, I⁻, BF₄⁻, PF₆⁻, and MeSO₃⁻.

2. The method of claim 1, wherein the transition metal complex is a compound of formula (IIa): 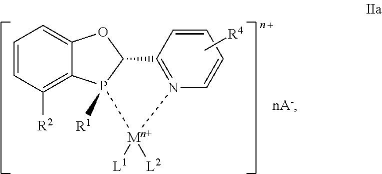
wherein
M is a transition metal selected from Rh and Ir;
A⁻ is a counter anion selected from the group consisting of chloro, bromo, halo, BF₄⁻, SbF₆⁻, TfO⁻, B(C₆H₅)₄⁻, B[3,5-(CF₃)₂C₆H₃]₄⁻, (BArF)⁻, or PF₆⁻;
n is the oxidation state of the transition metal M;
L¹ and L² are each olefins, or L¹ and L² together represent a diolefin;
R¹ is —(C₁-C₆)alkyl;
R² is H, —O(C₁-C₆)alkyl or —(C₁-C₆)alkyl; and
R⁴ is selected from —O(C₁-C₆)alkyl and —(C₁-C₆)alkyl.

3. The method of claim 2, wherein:
n is 1;
R¹ is t-Bu,
R² is —OCH₃;
R⁴ is —OCH₃ attached to the ortho-position of the pyridyl ring; and
L¹ and L² together represent a diolefin selected from norbornadiene and cyclooctadiene.

4. The method of claim 2, wherein M is Ir, and L¹ and L² together represent cyclooctadiene.

5. The method of claim 2, wherein A⁻ is Br⁻.

6. A method of making a diastereomeric salt of compound B (B-EA), 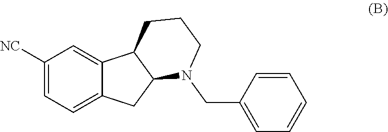 [SMILES: N#Cc1ccc2c(c1)[C@H]1CCCN(Cc3ccccc3)[C@H]1C2]
the process comprising reacting compound B with an enantiomeric organic acid to provide compound B-EA,
wherein the enantiomeric organic acid is selected from the group consisting of D-DTTA, D-DBTA, and D-tartaric acid.

7. A method of making compound A, 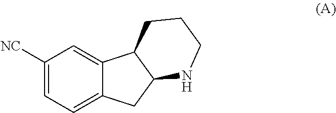 [SMILES: N#Cc1ccc2c(c1)[C@H]1CCCN[C@H]1C2]
the process comprising reacting compound B-EA from claim 5 with base followed by a reaction with a debenzylating reagent to provide compound A.

8. A method of making compound A, 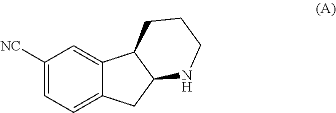 [SMILES: N#Cc1ccc2c(c1)[C@H]1CCCN[C@H]1C2]
the process comprising:
a) reacting compound C or C⁺X⁻ 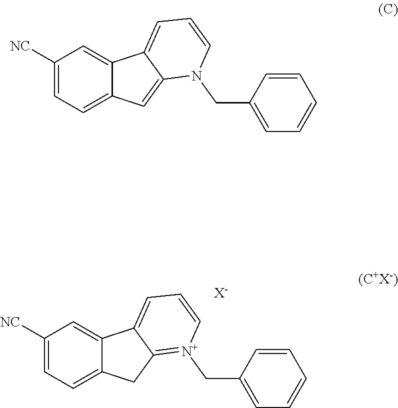
with a transition metal complex in the presence of hydrogen to provide compound B 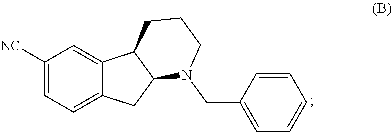 [SMILES: N#Cc1ccc2c(c1)[C@H]1CCCN(Cc3ccccc3)[C@H]1C2]
b) reacting compound B with an enantiomeric organic acid to provide compound B-EA, wherein the enantiomeric organic acid is selected from the group consisting of D-DTTA, D-DBTA, and D-tartaric acid; and
c) reacting compound B-EA with base followed by reaction with a debenzylating reagent to provide compound A.

9. A D-DTTA salt of compound B

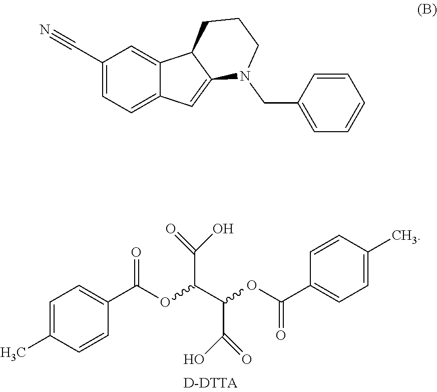

10. A method of making compound F: 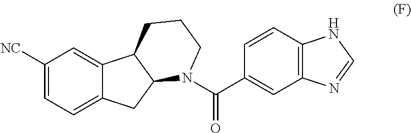 [SMILES: N#Cc1ccc2c(c1)[C@H]1CCCN(C(=O)c3ccc4[nH]cnc4c3)[C@H]1C2]
the method comprising:
preparing compound A according to claim 7; and
reacting compound A with 1H-benzoimidazole-5-carboxylic acid to provide compound F.

11. A method of making compound the hydrogen chloride (HCl) salt of compound F,
the method comprising,
preparing compound F according to claim 10; and
reacting compound F with hydrochloric acid in ethanol to provide the HCl salt of compound F.
